(19) United States
(12) Patent Application Publication
(10) Pub. No.: US 2003/0225048 A1
(43) Pub. Date: Dec. 4, 2003

(54) Sulfatase inhibiting continuous progestogen contraceptive regimens

(76) Inventors: Patrick Michel Caubel, Princeton, NJ (US); Andrew Joseph Friedman, Princeton, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/385,875

(22) Filed: Mar. 11, 2003

Related U.S. Application Data
(60) Provisional application No. 60/363,190, filed on Mar. 11, 2002.
(60) Provisional application No. 60/381,445, filed on May 17, 2002.

(51) Int. Cl. A61K 31/56; A61K 9/70

(52) U.S. Cl. 514/170; 424/449

(57) ABSTRACT

A method of contraception is described comprising the step of administering to a menstruating female a cycle of contraceptive therapy, said cycle of therapy including, for at most 35 successive days, the administration of a combination of an estrogen and a progestogen in a contraceptively effective daily dosage wherein said progestogen is a potent sulfatase inhibiting progestogen and said cycle of therapy including 4-8 days in which said progestogen is administered in the absence of estrogen administration following said at most 35 successive days.

The present invention relates to continuous progestogen contraceptive regimens for menstruating females. More particularly, the present invention relates to continuous progestogen contraceptive regimens employing a potent sulfatase inhibiting progestogen, such as, norgestimate (NGM) or norelgestromin (GMN), and an estrogen.

### BACKGROUND OF THE INVENTION

A substantial percentage of human breast carcinomas are hormone-dependent. Animal studies and clinical trials have confirmed that estrogens, particularly estradiol, are the most important hormones involved in supporting growth of hormone-dependent breast tumours. (see refs #1 at 493, #2 at 967, #7 at 1589, #8 at 525, #9 at 135, #10 at 225, #11 at 625 and #12 at 1497)

Plasma levels of estrone and estradiol in post-menopausal women are very low. (see refs #1 at 493 and #111 at 626) Yet, breast tumor tissue concentration of estrone and estradiol is an order of magnitude higher than plasma concentrations. (see refs #1 at 493, #2 at 967 and #13 at 641) FIG. 1 shows the enzymatic process by which estrogens are locally formed in human breast cancer cells and thereby made available to support growth. (see ref #10 at 229). Referring to FIG. 1, studies have shown that the sulfatase enzyme appears to be at least 10× more important in the formation of estrogens than the aromatase enzyme. (see refs #1 at 493, #2 at 967, #4 at 17, #5 at 931, #7 at 1589, #8 at 525, #9 at 135, #10 at 228, #11 at 626 and 628 and #13 at 641) Thus, it is the sulfatase pathway that is the primary pathway promoting local formation of estrogens in human breast cancer cells.

Since estradiol is one of the main factors involved in supporting growth of hormone-dependent breast tumours and the sulfatase pathway is the main pathway for the formation of estradiol in the breast, then a decrease of estradiol formation by suppression of the sulfatase pathway would have potential therapeutic activity in the management of breast cancer. (see refs #1 at 493, #3 at 55, #4 at 17, #5 at 931, #6 at 123 and #11 at 631) Suppression of the sulfatase pathway will have a breast protective effect.

It is an object of the present invention to provide a continuous progestogen contraceptive regimen to continuously suppress sulfatase activity in human breast cancer cells.

It is also an object of the present invention to provide a continuous progestogen contraceptive regimen with exceptional suppression of sulfatase activity in human breast cancer cells.

It is also an object of the present invention to provide a continuous progestogen contraceptive regimen to continuously suppress estrogen formation in human breast cancer cells.

It is yet another object of the present invention to provide a continuous progestogen contraceptive regimen with exceptional suppression of estrogen formation in human breast cancer cells.

It is still another object of the present invention to provide a continuous progestogen contraceptive regimen which minimizes exposure of the breast to locally formed estrogen.

It is another object of the present invention to provide a continuous progestogen contraceptive regimen which reduces exposure of the breast to estrogens as compared to other contraceptive regimens of equivalent estrogen dose.

It is another object of the present invention to provide a continuous progestogen contraceptive regimen with the lowest levels of breast estrogen exposure as compared to other contraceptive regimens of equivalent estrogen dose.

It is another object of the present invention to provide a continuous progestogen contraceptive regimen which closely limits exposure of the breast to those levels of estrogens which are administered in the regimen or produced in vivo outside the breast.

It is still another object of the present invention to provide a continuous progestogen contraceptive regimen which provides exceptional and continuous breast protective effect.

It is another object of the present invention to provide a continuous progestogen contraceptive regimen which minimizes risk factors associated with breast cancer.

### REFERENCES

1. Inhibition of Estrone Sulfatase Enzyme in Human Placenta and Human Breast Carcinoma; T. R. JEFFRY EVANS et al., J. Steroid Biochem. Molec. Biol. Vol. 39, No. 4A 1991, pp. 493-499.

2. In Vitro Effect of Synthetic Progestogens on Estrone Sulfatase Activity in Human Breast Carcinoma; ODILE PROST-AVALLET et al., J. Steroid Biochem. Molec. Biol., Vol. 39, No. 6, 1991, pp.967-973.

3. Effect of the progestagen R5020 (promegestone) and of progesterone on the uptake and on the transformation of estrone sulfate in MCF-7 and T-47d human mammary cancer cells: correlation with progesterone receptor levels; JORGE R. PASQUALINI et al., Cancer Letters, 66 (1992) 55-60, Elsevier Scientific Publishers Ireland Ltd.

4. Action of Danazol On The Conversion Of Estrone Sulfate To Estradiol And On The Sulfatase Activity In The MCF-7, T-47D and MDA-MB-231 Human Mammary Cancer Cells; B-L NGUYEN et al., J. Steroid Biochem, Molec. Biol. Vol. 46, No. 1, 1993, pp. 17-23.

5. Effect of Promegestone, Tamoxifen, 4-Hydroxytamoxifen and ICI 164,384 on the Oestrone Sulphatase Activity of Human Breast Cancer Cells; GERARD CHETRITE et al., Anticancer Research 13: 931-934 (1993).

6. Inhibition Of Steroid Sulfatase Activity By Danazol; KJELL CARLSTROM et al., Acta Obstet Gynecol Scand Suppl. 107-111.

7. Effect of the Progestagen Promegestone (R-5020) on mRNA of the Oestrone Sulphatase in the MCF-7 Human Mammary Cancer Cells; JORGE R. PASQUALINI et al., Anticancer Research 14: 1589-1594 (1994).

8. Effect of Nomegestrol Acetate on Estrone-sulfatase and 17β-Hydroxysteroid Dehydrogenase Activities in Human Breast Cancer Cells; G. CHETRITE et al., J. Steroid Biochem. Molec. Biol. Vol. 58, No.5/6, pp. 525-531, 1996.

9. Effect of Tibolone (Org OD14) and its Metabolites on Estrone Sulphatase Activity in MCF-7 and T-47D Mammary Cancer Cells; G. CHETRITE et al., Anticancer Research 17: 135-140 (1997).

10. Progestins and Breast Cancer; J. R. PASQUALINI et al., J. Steroid Biochem. Molec. Biol. Vol. 65, No. 1-6, pp.225-235, 1998.

11. Control of Estradiol In Human Breast Cancer. Effect of Medrogestone on Sulfatase, 17β-Hydroxysteroid Dehydrogenase And Sulfotransferase Activities in Human Breast Cancer Cells; JORGE RAUL PASQUALINI et al., Euro. Congr. On Menopause (1998), pp. 625-633.

12. Constitutive Expression of the Steroid Sulfatase Gene Supports the Growth of MCF-7 Human Breast Cancer Cells in Vitro and in Vitro; MATTIE R. JAMES et al., Endocrinology, Vol. 142, No.4, pp 1497-1505.

13. Concentrations of Estrone, Estradiol and Their Sulfates, And Evaluation of Sulfatase and Aromatase Activities in Patients with Breast Fibroadenoma; J. R. PASQUALINI et al., Int. J. Cancer, 70, pp. 639-643 (1997).

### SUMMARY OF THE INVENTION

According to the present invention there is provided, a method of contraception comprising the step of administering to a menstruating female a cycle of contraceptive therapy, said cycle of therapy including, for at most 35 successive days, the administration of a combination of an estrogen and a progestogen in a contraceptively effective daily dosage wherein said progestogen is a potent sulfatase inhibiting progestogen and said cycle of therapy including 4-8 days in which said progestogen is administered in the absence of estrogen administration following said at most 35 successive days.

There is also provided by the present invention, a contraceptive therapy unit for administration to a menstruating female comprising a cycle of separate dosage units, said cycle of dosage units including at most 35 dosage units adapted for successive daily oral administration, wherein said dosage units contain, in admixture with a pharmaceutically acceptable carrier, a combination of an estrogen and a progestogen in a contraceptively effective daily dosage wherein said progestogen is a potent sulfatase inhibiting progestogen and said cycle of dosage units including 4-8 dosage units adapted for successive daily oral administration containing said progestogen and no estrogen.

There is also provided by the present invention, a contraceptive therapy unit for administration to a menstruationg female comprising a cycle of transdermal patches, said cycle of transdermal patches including a sufficient number of patches adapted for successive administration to provide for at most 35 successive days of therapy, wherein said transdermal patches contain, in a suitable matrix, a combination of an estrogen and a progestogen for delivery in a contraceptively effective daily dosage wherein said progestogen is a potent sulfatase inhibiting progestogen and said cycle of transdermal patches including a patch for 4-8 successive days of therapy containing said progestogen and no estrogen.

There is also provided by the present invention, a contraceptive therapy unit for administration to a menstruationg female comprising a cycle of vaginal rings, said cycle of vaginal rings including a sufficient number of rings adapted for successive administration to provide for at most 35 successive days of therapy, wherein the vaginal rings contain, in a suitable matrix, a combination of an estrogen and a progestogen for delivery in a contraceptively effective daily dosage wherein said progestogen is a potent sulfatase inhibiting progestogen and said cycle of vaginal rings including a ring for 4-8 successive days of therapy containing said progestogen and no estrogen.

Applicants have surprisingly discovered that such a continuous progestogen regimen is expected to have reduced levels of estrogen in the breast as compared to other contraceptive regimens having equivalent doses of estrogens.

### BRIEF DESCRIPTION OF THE DRAWINGS

FIG. 1—Shows the enzymatic process involved in the formation and transformation of estrogens in human breast cancers.

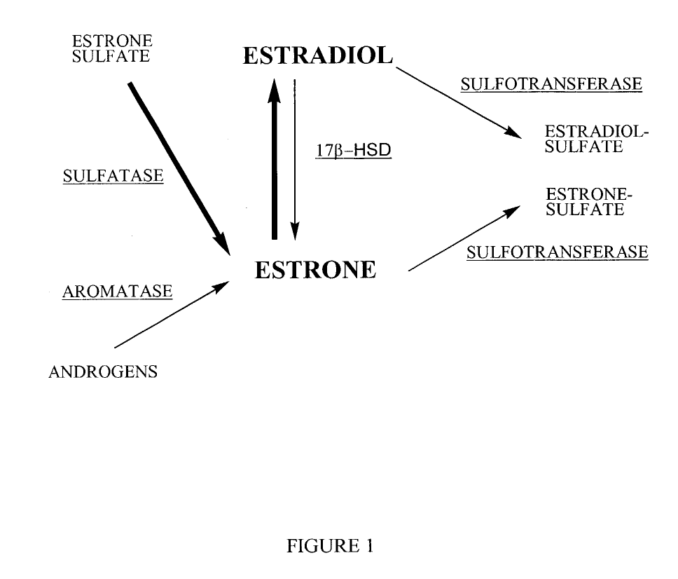

### DETAILED DESCRIPTION OF THE INVENTION

The contraceptive regimen according to the present invention is administered cycle after cycle to a menstruating female to achieve a long term contraceptive effect. Menstruating female is intended to refer to fertile women of child bearing age. The method of administration might be transdermal, vaginal or oral. Where administration is transdermal, a suitable patch is continuously worn with replacement as required. Where administration is vaginal, a suitable vaginal device, such as a ring, is continuously inserted with replacement as required. Where administration is oral, daily oral dosage units are administered.

Many common contraceptive regimens have a cycle of 28 days including 21 days of combined estrogen and progestogen administration followed by an off period of 7 days without administration of these hormones. In the off period, no hormone is administered to allow for menstruation. Continuous progestogen contraceptive regimen, as the phrase is used herein, is intended to refer to contraceptive regimens having combined estrogen and progestogen administration of 35 days or less followed by an off period of time with progestogen but without estrogen to allow menstruation. Thus, a maximum cycle would cover a period of time, which is 35 days of drug administration plus an off period of 4-8 days with progestogen but without estrogen. A preferred cycle is 21 days of combined estrogen and progestogen administration followed by an off period off 4-8 days, and most preferably 7 days, in which progestogen is administered without estrogen.

The withdrawal of estrogen administration during the off period will generally result in menstruation. It is a key aspect of the present invention to continue the administration of a potent sulfatase inhibiting progestogen during the off period to obtain a continuation of its breast protecting effect. Accordingly, it is desirable to continue progestogen administration for the full off period to the extent that a breast protecting effect is obtained and to the extent that such administration does not interfere with menstruation. The progestogen may be administered at a full dose or reduced dose. A full dose is intended to mean a continuation in the off period of the progestogen dose administered in the active period of the cycle or of a progestogen dose named below as suitable for administration in the active period of the cycle. A reduced or minimized dose might be a tablet delivered oral norgestimate equivalent dose of 30 or 60 mcg or device delivered systemic circulation norgestimate equivalent dose of 18 or 30 mcg. A preferred off period of time without estrogen administration to allow for menstruation is 7 days.

The continuous progestogen regimens herein may include a regimen in which there is a day to day or week to week variation in the dose of active administered according to a set pattern. In such a case the regimen, including variation of dose, is repeated in cycle following cycle. The continuous progestogen regimen may also be one in which there is no variation in the dose of the active administered. Whatever the case, a continuous progestogen contraceptive product utilizing the contraceptive regimen of the present invention is prescribed, sold and administered in units of cycles. The contraceptive product based on a cycle might be 2 to 6 vaginal rings that are inserted and then replaced every 7, 14 or 21 days according to their design. The contraceptive product based on a cycle might be 2 to 6 transdermal patches that are attached and then replaced every 7, 10 or 14 days according to their design. The contraceptive product based on a cycle might be 28, 35 or 42 tablets that are orally administered daily in a cycle that is 21/7, 28/7 or 35/7.

The estrogen in combination with the progestogen is administered in sufficient amounts to provide a contraceptive effect. Additionally, the estrogen dose in contraceptive regimens described herein is closely associated with the control of bleeding and spotting in the cycle. Between menstruations, bleeding and spotting should be minimized. Thus, 17α-ethinylestradiol might be also administered in sufficient amounts to control or minimize or eliminate bleeding and spotting during the inter-menstruation period of the cycle.

“Estrogen” herein refers to an estrogen receptor modulator having either an agonistic or antagonistic effect on the estrogen receptor, but preferably an agonistic effect. Any conventional estrogen may be employed as a suitable component in the contraceptive regimen of this invention. The particular estrogen employed should be selected and administered such that it is equivalent in contraceptive effect to a daily dosage of about 0.005-0.050 mg of 17α-ethinylestradiol. The preferred dosage of the estrogen employed is one equal to a daily dosage of about 0.010-0.035 mg of 17α-ethinylestradiol.

In addition to the commonly employed 17β-estradiol, there can be also be employed 17α-ethinylestradiol, esters and ethers of 17α-ethinylestradiol such as, for example, 17α-ethinylestradiol 3-dimethylamino propionate, 17α-ethinylestradiol 3-cyclopentyl ether (quienestrol) and 17α-ethinylestradiol 3-methyl ether (mestranol) as the estrogen component. Natural estrogens such as estrone, estrone sulfate, estrone sulfate piperazine salt, estradiol and estriol, and their esters, may also be employed. Conjugated equine estrogens (CEE) or conjugated estrogens (CE) are well known for this use. Suitable synthetic estrogens or synthetic estrogen modulators for use herein include tamoxifen, toremifene, ormeloxifene, modrefen, fulvestrant, lasofoxifene, bazedoxifene (TSE-424), arzoxifene, tesmilifene, miproxifene, EM-652 (Sch-57068), 3339 (Aventis), Ospemifene (Fc 1271A), ERA-923, GTx-006, HM-101, DPC-974, A-007, SP-8490, WAY-140424, tibolone, levodoxiphen, raloxifene.

In the case of a daily oral tablet, there is administered a preferred dose of 17α-ethinylestradiol (or contraceptively equivalent amount of a suitable estrogen) between about 0.005 mg to about 0.050 mg and more preferably between about 0.010 mg to about 0.035 mg. Specific daily oral tablets might contain 0.015, 0.020, 0.025 or 0.035 mg of 17α-ethinylestradiol. In the case of a vaginal ring, the preferred ring delivers to systemic circulation a daily dose of 17α-ethinylestradiol (or contraceptively equivalent amount of a suitable estrogen) between about 0.003 mg to about 0.030 mg and more preferrably between about 0.006 mg to about 0.020 mg. A specific vaginal ring might be inserted for one week and deliver to systemic circulation in that period of time an average daily dose of 0.009, 0.012, 0.015 or 0.020 mg of 17α-ethinylestradiol. In the case of a transdermal patch, a preferred patch delivers to systemic circulation a daily dose of 17α-ethinylestradiol (or contraceptively equivalent amount of a suitable estrogen) between about 0.003 mg to about 0.030 mg and more preferrably between about 0.006 mg to about 0.020 mg. A specific patch might be worn for one week and deliver to the surface of the skin in that period of time an average daily dose of 0.009, 0.012, 0.015 or 0.020 mg of 17α-ethinylestradiol. Regardless of the foregoing, it is intended herein to use conventional amounts of estrogen since it is not the estrogen component which is critical to the invention. Persons skilled in the art well understand required doses of estrogen required in contraceptive regimens.

A potent sulfatase inhibiting progestogen is preferably herein defined as a progestogen which has (or a progestogen with a substantial metabolite thereof which has) an IC₅₀ in the conversion of E₁S to E₂ in either the MCF-7 or T-47D breast cancer cell lines of about the corresponding IC₅₀ of norelgestromin or lower. A potent sulfatase inhibiting progestogen may also be a progestogen which has (or a progestogen with a substantial metabolite thereof which has) an IC₅₀ in the conversion of E₁S to E₂ in either the MCF-7 or T-47D breast cancer cell lines of substantially less than the corresponding IC₅₀ of medroxyprogesterone acetate, for example, on the order of ⅓, ½ or ⅕ of the IC₅₀ of medroxyprogesterone acetate. A potent sulfatase inhibiting progestogen can also be defined as a progestogen having (or a progestogen with a substantial metabolite thereof which has) an IC₅₀ in the conversion of E₁S to E₂ in either the MCF-7 or T-47D breast cancer cell lines of at most about 1/10, or about preferably 1/100, the corresponding IC₅₀ of medroxyprogesterone acetate (MPA). A potent sulfatase inhibiting progestogen can also be defined as a progestogen which inhibits (or a progestogen with a substantial metabolite thereof which inhibits) at least about 70% and preferably at least about 90% of the conversion of E₁S to E₂ in either the MCF-7 or T-47D breast cancer cell lines where employed in the test at a concentration of 50×10⁻⁶ mol/l.

Norgestimate (NGM) or norelgestromin (NGMN) are the preferred progestogens utilized herein and are each known to the art of contraceptive therapy. In fact, norgestimate is now used in a number of commercially available contraceptive products. The most preferred progestogen is norelgestromin (17-d-norgestimate). Norelgestromin is the major metabolite of norgestimate in humans with 80% and higher of norgestimate being converted to norelgestromin in vivo. For this reason, inhibition of sulfatase enzyme activity which is demonstrated for norelgestromin is inferred to norgestimate. Of course, to obtain equivalent inhibition of sulfatase enzyme activity (but not progestogenic effect), it may be necessary to administer a somewhat greater dose of norgestimate as compared to any dose of norelgestromin.

The progestogen is administered in conjunction with the estrogen in an amount sufficient to produce a contraceptive effect. The progestogen will also oppose the action of the estrogen on the endometrium. It has been observed that the long term administration of an estrogen which is unopposed by the administration of a progestogen leads to a substantial increase in the incidence of endometrial cancer. Thus, it is also desirable in a contraceptive regimen that the progestogen be administered in an amount which is an effective endometrium protective amount.

According to the present invention, it is now an additional requirement that the progestogen be administered in an amount which is an effective breast protective amount. More specifically, in a first characterization of a breast protective and otherwise suitable amount of progestogen, there is selected and administered sufficient sulfatase inhibiting progestogen such that it is at least equivalent in both contraceptive and breast protecting effect to about 0.030 mg to about 0.500 mg of orally administered norgestimate. Preferably, there is selected and administered sufficient sulfatase inhibiting progestogen such that it is at least equivalent in both contraceptive and breast protecting effect to about 0.050 mg to about 0.300 mg of orally administered norgestimate. In another characterization of a breast protective amount of progestogen and assuming a contraceptively effective amount, there is administered sufficient active compound to provide for, during a substantial portion of each day, a substantial supression of sulfatase activity, for example, of 50% or greater and preferably of 67% or greater and most preferably of 75% or greater. A substantial portion of a day is intended to mean a period of at least 4 hours, but within the invention might mean a period of at least 8 hours or 12 hours or even 24 hours. In the case of a daily oral tablet, there is administered a preferred dose of norgestimate or norelgestromin (or contraceptively equivalent amount of a suitable progestogen) between about 30 mcg to about 500 mcg and more preferably between about 50 mcg to about 300 mcg. Specific daily oral tablets might contain 125, 180, 215, 250 or 300 mcg of norgestimate or norelgestromin. In the case of a vaginal ring, a preferred ring delivers to systemic circulation a daily dose of norgestimate or norelgestromin (or contraceptively equivalent amount of a suitable progestogen) between about 18 mcg to about 300 mcg and more preferrably between about 30 mcg to about 175 mcg. A specific vaginal ring might be inserted for one week and deliver to systemic circulation in that period of time an average daily dose of 70, 100, 125, 140 or 175 mcg of norgestimate or norelgestromin. In the case of a transdermal patch, a preferred patch delivers to systemic circulation a daily dose of norgestimate or norelgestromin (or contraceptively equivalent amount of a suitable progestogen) between about 18 mcg to about 300 mcg and more preferrably between about 30 mcg to about 175 mcg. A specific patch might be worn for one week and deliver to systemic circulation in that period of time an average daily dose of 70, 100, 125, 140 or 175 mcg of norgestimate or norelgestromin.

In Table 1, there are disclosed preferred oral daily continuous progestogen contraceptive regimens according to the present invention containing norgestimate (NGM) or norelgestromin (NGMN) and 17α-ethinylestradiol (EE). A placebo containing no hormone is administered in the off period and a single tablet is administered in the active period containing the hormones as reported.

TABLE 1

| | | Active tablet NGM or | |
|---|---|---|---|
| | Active/ | NGMN/EE content | Off tablet NGM or |
| Regimen # | Off days | (mcg) | NGMN content (mcg) |
| 1 | 21/7 | 125/20 | 125 |
| 2 | 21/7 | 180/20 | 180 |
| 3 | 21/7 | 250/20 | 250 |
| 4 | 21/7 | 125/25 | 125 |
| 5 | 21/7 | 180/25 | 180. |
| 6 | 21/7 | 250/25 | 250 |
| 7 | 21/7 | 122/35 | 125 |
| 8 | 21/7 | 180/35 | 180 |
| 9 | 21/7 | 250/35 | 250 |
| 10 | 21/7 | 7 days 180/20 then | 180 |
| | | 7 days 215/20 then | |
| | | 7 days 250/20 | |
| 11 | 21/7 | 7 days 180/25 then | 180 |
| | | 7 days 215/25 then | |
| | | 7 days 250/25 | |
| 12 | 21/7 | 7 days 180/35 then | 180 |
| | | 7 days 215/35 then | |
| | | 7 days 250/35 | |
| 13 | 21/7 | 250/20 | 180 |
| 14 | 21/7 | 250/25 | 180 |
| 15 | 21/7 | 250/35 | 180 |
| 16 | 21/7 | 7 days 180/20 then | 250 |
| | | 7 days 215/20 then | |
| | | 7 days 250/20 | |
| 17 | 21/7 | 7 days 180/25 then | 250 |
| | | 7 days 215/25 then | |
| | | 7 days 250/25 | |
| 18 | 21/7 | 7 days 180/35 then | 250 |
| | | 7 days 215/35 then | |
| | | 7 days 250/35 | |
| 19 | 21/7 | 7 days 180/20 then | 125 |
| | | 7 days 215/20 then | |
| | | 7 days 250/20 | |
| 20 | 21/7 | 7 days 180/25 then | 125 |
| | | 7 days 215/25 then | |
| | | 7 days 250/25 | |
| 21 | 21/7 | 7 days 180/35 then | 125 |
| | | 7 days 215/35 then | |
| | | 7 days 250/35 | |
| 22 | 21/7 | 7 days 180/20 then | 50 |
| | | 7 days 215/20 then | |
| | | 7 days 250/20 | |
| 23 | 21/7 | 7 days 180/25 then | 50 |
| | | 7 days 215/25 then | |
| | | 7 days 250/25 | |
| 24 | 21/7 | 7 days 180/35 then | 50 |
| | | 7 days 215/35 then | |
| | | 7 days 250/35 | |
| 25 | 21/7 | 250/20 | 125 |
| 26 | 21/7 | 250/25 | 125 |
| 27 | 21/7 | 250/35 | 125 |
| 28 | 21/7 | 250/20 | 50 |
| 29 | 21/7 | 250/25 | 50 |
| 30 | 21/7 | 250/35 | 50 |

In Table 2, there are disclosed preferred contraceptive transdermal regimens or vaginal ring regimens according to the present invention using weekly patches or rings containing norgestimate (NGM) or norelgestromin (NGMN) and 17α-ethinylestradiol (EE). The weekly patches or rings deliver to systemic circulation the reported average daily dose of NGM or NGMN or EE.

TABLE 2

| | | Active Device NGM or | Off Device NGM or |
|---|---|---|---|
| | Active/Off | NGMN/EE delivery | NGMN delivery rate |
| Regimen # | weeks | rate (mcg) | (mcg) |
| 31 | 3/1 | 70/12 | 70 |
| 32 | 3/1 | 100/12 | 100 |
| 33 | 3/1 | 140/12 | 140 |
| 34 | 3/1 | 70/15 | 70 |
| 35 | 3/1 | 100/15 | 100 |
| 36 | 3/1 | 140/15 | 140 |
| 37 | 3/1 | 70/20 | 70 |
| 38 | 3/1 | 100/20 | 100 |
| 39 | 3/1 | 140/20 | 140 |
| 40 | 3/1 | 1 week 100/12 then | 100 |
| | | 1 week 120/12 then | |
| | | 1 week 140/12 | |
| 41 | 3/1 | 1 week 100/15 then | 100 |
| | | 1 week 120/15 then | |
| | | 1 week 140/15 | |
| 42 | 3/1 | 1 week 100/20 then | 100 |
| | | 1 week 120/20 then | |
| | | 1 week 140/20 | |
| 43 | 3/1 | 140/12 | 100 |
| 44 | 3/1 | 140/15 | 100 |
| 45 | 3/1 | 140/20 | 100 |
| 46 | 3/1 | 1 week 100/12 then | 140 |
| | | 1 week 120/12 then | |
| | | 1 week 140/12 | |
| 47 | 3/1 | 1 week 100/15 then | 140 |
| | | 1 week 120/15 then | |
| | | 1 week 140/15 | |
| 48 | 3/1 | 1 week 100/20 then | 140 |
| | | 1 week 120/20 then | |
| | | 1 week 140/20 | |
| 49 | 3/1 | 1 week 100/12 then | 70 |
| | | 1 week 120/12 then | |
| | | 1 week 140/12 | |
| 50 | 3/1 | 1 week 100/15 then | 70 |
| | | 1 week 120/15 then | |
| | | 1 week 140/15 | |
| 51 | 3/1 | 1 week 100/20 then | 70 |
| | | 1 week 120/20 then | |
| | | 1 week 140/20 | |
| 52 | 3/1 | 1 week 100/12 then | 30 |
| | | 1 week 120/12 then | |
| | | 1 week 140/12 | |
| 53 | 3/1 | 1 week 100/15 then | 30 |
| | | 1 week 120/15 then | |
| | | 1 week 140/15 | |
| 54 | 3/1 | 1 week 100/20 then | 30 |
| | | 1 week 120/20 then | |
| | | 1 week 140/20 | |
| 55 | 3/1 | 140/12 | 70 |
| 56 | 3/1 | 140/15 | 70 |
| 57 | 3/1 | 140/20 | 70 |
| 58 | 3/1 | 140/12 | 30 |
| 59 | 3/1 | 140/15 | 30 |
| 60 | 3/1 | 140/20 | 30 |

The estrogen and progestogen component are orally administered preferably together in tablets also containing a pharmaceutically acceptable non-toxic carrier, but they can also be administered separately. Suitable carriers include magnesium carbonate, magnesium stearate, talc, lactose, sugar, peptin, dextrin, starch, methylcellulose, sodium carboxylmethylcellulose, and the like. The tablet may also contain one or more substances, which act as diluents, flavoring agents, solubilizers, lubricants, suspending agents, binders, or tablet disintegrating agents as well as encapsulating materials. In general, the active agents are processed, together with the usual additives, vehicles and/or flavor-ameliorating agents normally employed in Galenic pharmacy, in accordance with generally accepted pharmaceutical practices. The hormone containing tablets might also contain nutritional supplements such as, for example, iron supplements, folic acid, calcium, vitamin B₆, vitamin B₁₂, etc. In the manufacture of a typical tablet, the active agents are granulated with spray dried lactose, a lubricating agent and a colorant and compressed.

Oral tablets are preferably packaged in the form of a pharmaceutical kit or package in which the daily dosages are arranged for proper sequential administration. This invention also relates, therefore, to a pharmaceutical unit which contains the tablets of the regimen in a synchronized, fixed sequence, wherein the sequence or arrangement of the dosage units corresponds to the regimen of daily administration.

The estrogen and progestogen component may be transdermally administered, preferably together, by use of a patch. Broadly, patches are devices which contain at a minimum a drug reservoir matrix for holding the drug and metering the drug deposition or delivery to the skin, a backing, and an adhesive layer for adhering the device to the patient. The device may contain other layers such as a drug release rate controlling layer for modulating delivery rate, and the like. The device may contain permeation enhancers to increase the rate of penetration of drugs across the skin. Patches are well known and understood by persons skilled in the art. Patches are now employed in marketed products for the administration of certain progestogens and estrogens. Specific patches and even their application to steroids of the type described herein are described in U.S. Pat. Nos. 5,474,783; 5,656,286; 5,958,446; 6,024,976; 5,252,334; 5,006,342; and 4,906,463.

The estrogen and progestogen component may be intravaginally administered, preferably together, by use of a ring. Broadly, rings are devices having an elastomeric portion or body into which the active steroid is dispersed and which acts as a reservoir and meter for the diffusion of active to the lining of the vagina. The ring may be composed entirely of elastomer with steroid homogenously dispersed throughout as described in U.S. Pat. No. 3,545,397. The ring may have an inert inner core surrounded by an active containing elastomeric layer as described in U.S. Pat. No. 4,012,496. The ring may have an elastomeric active containing inner core surrounded by a thin elastomeric layer initially containing no active. The ring may have an inert core, surrounded by an active containing elastomeric layer and further surrounded by an elastomeric outer layer of variable thickness initially containing no active as described in U.S. Pat. No. 4,292,965. The elastomer, the layered design of the ring, its surface area, the concentration of active, the nature of the active, etc. all combine to determine the release rate of active. Rings are well known and understood by persons skilled in the art. Rings are now employed in marketed products for the administration of certain steroids. Further specific rings and their application to steroids of the type described herein are described in U.S. Pat. Nos. 4,871,543 and 5,188,835.

### Biological Test Methods

Chemicals

[6,7⁻³H(N)]-estrone sulfate (³H-E₁S), ammonium salt (sp. act. 53 Ci/mmol) and [4-¹⁴C]-estradiol (¹⁴C-E₂) (sp. act. 57 mCi/mmol) were purchased from New England Nuclear Division (DuPont de Nemours, Les Ulls, France). The purity of the radioisotopes was assessed by thin-layer chromatography (TLC) in the appropriate system before use. E₁S, ammonium salt, unlabeled E₁ and E₂, (analytical grade) were obtained from Sigma-Aldrich Chimle, (St Quentin Fallavier, France). 17-deacetylnorgestimate (NGMN; 13-ethyl-17-hydroxy-18,19-dinor-17α-pregn-4-en-20-yn-3-one oxime) was a gift from R. W. Johnson Pharmaceutical Research Institute, Medicinal Chemistry Department, (Raritan, N.J., USA); medroxyprogesterone acetate (MPA, 17α-acetoxy-6α-methylprogesterone) was obtained from Sigma-Aldrich Chimie. All other chemicals were of the highest grade commercially available.

Cell Culture

The hormone-dependent MCF-7 and T-47D human mammary cancer cell lines were grown in Eagle's Minimal Essential Medium (MEM) buffered with 10 mmol/l HEPES (pH 7.6), supplemented with 2 mmol/1 L-glutamine, 100 U/ml penicillin-streptomycin and 5% fetal calf serum (FCS) (A.T.G.C., Marne-la-Vallee, France) for T-47D, or 10% FCS for MCF-7 cells, and incubated at 37° C. n a humidified atmosphere of 5% CO₂. Media were changed twice a week. The cells were passed every 10-12 days and replated in 75 cm² flasks (A.T.G.C.) at 3×10⁶ cells/flask. Four days before the experiments, the cells were transferred to MEM containing 5% steroid-depleted treated FCS. The FCS had been treated overnight at 4° C. with dextran-coated charcoal (DCC)(0.1-1% w/v, DCC-FCS). The MCF-7 and T-47D cell lines used herein were deposited in accordance with the Budapest Treaty under the references MCF7_JJPRD and T47D_JJPRD on May 17, 2002 at The Belgian Co-ordinated Collections of Microorganisms (BCCM), Laboratorium voor Moleculaire Biologie, Universiteit Gent, K. L. Ledeganckstraat 35, B-9000 Gent, Belgium and are publicly available under accession numbers LMBP 5862CB and LMBP 5863CB, respectively. Isolation and quantification of [³H]-estradiol from human mammary cancer cells incubated with [³H]-E₁S

Preconfluent cells were incubated for 4 hours at 37° C. in MEM-DCC-FCS with the addition of 5×10⁻⁹ mol/l of [³H]-E₁S, alone (control cells) or in combination with the different compounds: NGMN or MPA, dissolved in ethanol (final concentration <0.2%), at a range of concentrations of 5×10⁻⁵-5×10⁻⁹ mol/l. Control cells received ethanol vehicle only. After 24 hours, the medium was removed, the cells washed twice with ice-cold Hank's Buffered Saline Solution (HBSS, calcium-magnesium-free)(A.T.G.C.) and harvested by scraping. After centrifugation, the pallet was treated with 80% ethanol and the radioactivity extracted for at least 24 h at 20° C. The cellular radioactivity uptake was determined in the ethanolic supernatant and the DNA content in the remaining pellet was evaluated according to Burton Biochem Journal 62:315-323, 1956. [¹⁴C]-E₂ (5,000 dpm) was added to monitor analytical losses and unlabeled E₁ and E₂ (50 μg) were used as carriers and reference indicators. In the total ethanolic extracts, E₂ was isolated by thin layer chromatography (TLC) on silica gel 60F254 (Merck, Darmstadt, Germany), developed with chloroform-ethylacetate (4:1, v/v) system. After visualization of the estrogens under U.V. at 254 nm, the appropriate areas were cut off into small pieces, placed in liquid scintillation vials with ethanol (0.5 ml) and allowed to extract for 30 nm. Three ml of Opti-fluor (Packard, Rungis, France) were added and the vials were analyzed for ³H and ¹⁴C contents with quench correction by external standarization. The quantitative evaluation of E₂ was calculated as a percentage of the total radioactivity associated with the cells and then expressed as fmol of E₂ formed/mg DNA from E₁S.

Statistical Analysis

Data are expressed as the mean±standard error of the mean (SEM) values. Student's t-test was used to assess the significance of the differences between means; p values≦0.05_were considered significant.

### Results

Table 3 shows the effects of NGMN and medroxyprogesterone acetate (MPA) concentrations on the conversion of E₁S to E₂ in the hormone-dependent human breast cancer cell line T-47D The data are the mean±SEM of duplicate determinations of 3 independent experiments. * p≦0.05 vs contol values (non-treated cells); ** p≦0.005 vs contol values (non-treated cells)

TABLE 3
T-47D

| NGMN or MPA | NGMN | MPA |
|---|---|---|
| conc 1 × 10⁻⁶ | E₂ formed fmol/mg DNA | E₂ formed fmol/mg DNA |
| mol/l | (% inhibition) | (% inhibition) |
| 0 (control) | 1805 ± 152 (0%) | |
| 0.005 | 1029 ± ? (43 ± 7%)* | 1245 ± ? (31 ± 5%)* |
| 0.5 | 469 ± ? (74 ± 4%)* | 957 ± ? (47 ± 3%)* |
| 50 | 54 ± ? (97 ± 2%)** | 704 ± ? (61 ± 3%)* |

Table 4 shows the effects of NGMN and medroxyprogesterone acetate (MPA) concentrations on the conversion of E₁S to E₂ in the hormone-dependent human breast cancer cell line MCF-7. The data are the mean±SEM of duplicate determinations of 3 independent experiments. * p≦0.05 vs contol values (non-treated cells); ** p≦0.005 vs contol values (non-treated cells)

TABLE 4
MCF-7

| NGMN or MPA | NGMN | MPA |
|---|---|---|
| conc 1 × 10⁻⁶ | E₂ formed fmol/mg DNA | E₂ formed fmol/mg DNA |
| mol/l | (% inhibition) | (% inhibition) |
| 0/control | 2185 ± 101 (0%) | |
| 0.005 | 1639 ± ? (25 ± 4%)* | 2054 ± ? (6 ± 3%) |
| 0.5 | 940 ± ? (57 ± 5%)* | 1748 ± ? (20 ± 3%) |
| 50 | 87 ± ? (96 ± 2%)** | 808 ± ? (63 ± 4%)* |

Table 5 shows the IC₅₀ values for NGMN and medroxyprogesterone acetate (MPA) in the conversion of E₁S to E₂ in the hormone-dependent human breast cancer cell lines MCF-7 and T-47D. IC₅₀ values correspond to the 50% inhibition of the conversion of E₁S to E₂ and were determined using non-linear regression analysis.

TABLE 5

IC₅₀, 1 × 10⁻⁶ mol/l

| | T-47D | MCF-7 |
|---|---|---|
| NGMN | 0.0127 | 0.178 |
| MPA | 2.15 | 26.1 |

Having described the invention in specific detail and exemplified the manner in which it may be carried into practice, it will be apparent to those skilled in the art that imnumerable variations, applications, modifications, and extensions of the basic principles involved may be made without departing from its spirit or scope. It is to be understood that the foregoing is merely exemplary and the present invention is not to be limited to the specific form or arrangements of parts herein described and shown.

What is claimed is:

1. A method of contraception comprising the step of administering to a menstruating female a cycle of contraceptive therapy, said cycle of therapy including, for at most 35 successive days, the administration of a combination of an estrogen and a progestogen in a contraceptively effective daily dosage wherein said progestogen is a potent sulfatase inhibiting progestogen and said cycle of therapy including 4-8 days in which said progestogen is administered in the absence of estrogen administration following said at most 35 successive days.

2. A contraceptive therapy unit for administration to a menstruationg female comprising a cycle of separate dosage units, said cycle of dosage units including at most 35 dosage units adapted for successive daily oral administration, wherein said dosage units contain, in admixture with a pharmaceutically acceptable carrier, a combination of an estrogen and a progestogen in a contraceptively effective daily dosage wherein said progestogen is a potent sulfatase inhibiting progestogen and said cycle of dosage units including 4-8 dosage units adapted for successive daily oral administration containing said progestogen and no estrogen.

3. There is also provided by the present invention, a contraceptive therapy unit for administration to a menstruationg female comprising a cycle of transdermal patches, said cycle of transdermal patches including a sufficient number of patches adapted for successive administration to provide for at most 35 successive days of therapy, wherein said transdermal patches contain, in a suitable matrix, a combination of an estrogen and a progestogen for delivery in a contraceptively effective daily dosage wherein said progestogen is a potent sulfatase inhibiting progestogen and said cycle of transdermal patches including a patch for 4-8 successive days of therapy containing said progestogen and no estrogen.

4. There is also provided by the present invention, a contraceptive therapy unit for administration to a menstruating female comprising a cycle of vaginal rings, said cycle of vaginal rings including a sufficient number of rings adapted for successive administration to provide for at most 35 successive days of therapy, wherein the vaginal rings contain, in a suitable matrix, a combination of an estrogen and a progestogen for delivery in a contraceptively effective daily dosage wherein said progestogen is a potent sulfatase inhibiting progestogen and said cycle of vaginal rings including a ring for 4-8 successive days of therapy containing said progestogen and no estrogen.
